(19) United States
(12) Patent Application Publication
(10) Pub. No.: US 2019/0330497 A1
(43) Pub. Date: Oct. 31, 2019

(54) REMOVABLE ADHESIVE FOR DEVICE COVERINGS

(71) Applicant: Precision Dynamics Corporation, Valencia, CA (US)

(72) Inventor: Richard Kapusniak, Milwaukee, WI (US)

(73) Assignee: Precision Dynamics Corporation, Valencia, CA (US)

(21) Appl. No.: 15/963,934

(22) Filed: Apr. 26, 2018

(51) Int. Cl. C09J 7/38; C09J 7/24; C09J 7/25; C09J 5/00; A61B 46/10; A61B 6/00

(52) U.S. Cl. CPC C09J 7/385; C09J 7/383; C09J 7/243; C09J 7/255; C09J 5/00; A61B 5/055; A61B 6/4423; C09J 2433/00; C09J 2421/00; C09J 2423/046; C09J 2467/006; A61B 46/10

(57) ABSTRACT

The present disclosure provides example differential adhesive systems, which may be used with various device covers. An example differential adhesive comprises an intermediary body having a first side and an opposite second side, a first adhesive layer attached to the first side, and a second adhesive layer attached to the second side. The first adhesive layer comprises an acrylic adhesive, and the second adhesive layer comprises a rubber adhesive secured to the plastic article. In some embodiments, the intermediary body comprises a polyester film. The acrylic adhesive of the first adhesive layer may comprise a cross-linked pressure sensitive acrylic adhesive and include a peel adhesion of approximately 40 ounces per inch to approximately 60 ounces per inch. The first adhesive layer is may be configured for releasable attachment to the surface of the first object. The second adhesive layer may be configured for permanent attachment to the plastic article.

### TECHNICAL FIELD

The present disclosure generally relates to adhesive systems and more specifically to an adhesive system for device coverings.

### BACKGROUND

A plastic article is often applied over devices to serve as a protective barrier for prolonged, but temporary periods of time. Such plastic articles are often large plastic coverings used to protect items during a temporary process. For example, to protect equipment during a procedure, or components during product manufacture or maintenance.

A cover is often applied between a patient and a device to serve as a protective barrier to fluids for the device or to create a sterile barrier. Existing covers are difficult to place by a single gloved professional, difficult to fit well and problematic to remove. Existing covers also leave adhesive residues on a device, especially in practice where operational realities mean that covers are allowed to dwell on a device for hours or days between uses. Over time, such residues will collect dust and dirt which is not desired in a setting, and which typically require the use of solvents that may be harmful and toxic to humans and require proper storage safety.

Effective adhesive solutions for plastic protective barriers should, therefore, not only support a plastic barrier on a surface for a prolonged but temporary period of time, the adhesive solution should also be cleanly removable without tearing the plastic barrier or leaving residue on the surface. The above identified technical problems are reduced or eliminated by the systems and methods disclosed in the present disclosure.

### SUMMARY

Embodiments of differential adhesive systems, as well as method and computer executable instructions for manufacturing the differential adhesive systems are provided in the present disclosure.

In one aspect, which may include at least a portion of the subject matter of any of the preceding and/or following examples and aspects, a differential adhesive for securing a plastic article to a surface of a first object is described. The differential adhesive, in some implementations, comprises: an intermediary body having a first side and an opposite second side, a first adhesive layer attached to the first side, and a second adhesive layer attached to the second side. The first adhesive layer comprises an acrylic adhesive, and the second adhesive layer comprises a rubber adhesive secured to the plastic article.

In some embodiments, the intermediary body comprises a polyester film. In other embodiments, the intermediary body comprises a polyethylene film.

The acrylic adhesive of the first adhesive layer may include a peel adhesion of approximately 40 ounces per inch to approximately 60 ounces per inch. The acrylic adhesive of the first adhesive layer may comprise a cross-linked pressure sensitive acrylic adhesive. The first adhesive layer may be configured for releasable attachment to the surface of the first object. The second adhesive layer may be configured for permanent attachment to the plastic article.

The plastic article may be a device covering comprising polyethylene. In some embodiments, the plastic article is radiolucent. In some embodiments, the first object is a CT scanner.

Other implementations of this disclosure include corresponding devices, systems, and computer programs, as well as and associated methods for operating and manufacturing the described devices and systems. These other implementations may each optionally include one or more of the following features. For instance, provided is a device cover comprising a covering of plastic material and a differential adhesive for securing the covering to a surface of a first object. The differential adhesive comprises an intermediary body having a first side and an opposite second side. The first adhesive layer is attached to the first side and may comprise an acrylic adhesive. The second adhesive layer is attached to the second side and may comprise a rubber adhesive secured to the covering. The covering may comprise a polyethylene film.

Further described is a method of attaching a device cover to a surface of a device. The method comprises removing a liner from a differential adhesive secured to the device cover. As described, the differential adhesive comprises an intermediary body having a first side and an opposite second side, a first adhesive layer attached to the first side, and a second adhesive layer attached to the second side. The first adhesive layer may comprise an acrylic adhesive covered by the liner, and the second adhesive layer may comprise a rubber adhesive secured to the covering. The method further comprises applying the exposed first adhesive layer to the surface of the device.

Further described is a non-transitory computer readable medium comprising computer executable instructions stored thereon, which, when executed by one or more computers, cause a machine to manufacture a differential adhesive system as described in any of the implementations above.

These and other embodiments are described further below with reference to the figures.

### BRIEF DESCRIPTION OF THE DRAWINGS

FIGS. 1A and 1B illustrate an enlarged fragmentary vertical section of a differential adhesive system in accordance with various embodiments of the present disclosure.

FIGS. 2A and 2B illustrate an example device cover in accordance with various embodiments of the present disclosure.

FIG. 3 illustrates an example method for attaching a device cover to a surface of a device in accordance with various embodiments of the present disclosure.

Embodiments of the present disclosure and their advantages are best understood by referring to the detailed description that follows. Like reference numbers identify like elements illustrated in one or more of the figures.

### DETAILED DESCRIPTION

The present disclosure provides example adhesives and device covers that can be implemented with various embodiments of the present disclosure.

### Overview

Often devices require protective covers to create a protective barrier to fluids for the device, or to create a sterile barrier between a patient and the device. Such covers may maintain sterile or hygienic standard in a clinical setting, as well as protect the equipment from bodily fluids and spills and leakages. Such devices may include imaging equipment used by hospitals and imaging centers, such as Computed Tomography (CT) machines or Magnetic Resonance Imaging (MRI) machines. Such devices may include surfaces comprising painted metal or plastic, and which may be textured.

Existing covers implement either a rubber adhesive or an acrylic adhesive. Such existing covers are difficult to place by one person, difficult to fit, and problematic to remove. For example, existing covers have to be removed shortly after use to be cleanly removed. Otherwise, adhesive residues may remain on the device after removal. Over time, any residual adhesive will collect dust and dirt which is not desired in a clinical setting. Residual adhesive removal typically requires the use of solvents that are harmful and toxic and require proper storage safety.

Typically device covers are fabricated from plastic, such as polyethylene film, which includes a low surface energy. Most pressure sensitive adhesives (PSAs) cannot functionally hold such plastic covers onto the surface of a device. Acrylic PSAs preferentially adhere to the device instead of the polyethylene cover due to low bond strength with polyethylene film. Rubber PSAs generally include stronger bond strengths to polyethylene film, as well high bond strength to device surfaces. As a result, rubber PSAs require a high peel force to remove which can cause the polyethylene cover to tear, or which can leave residual adhesive on the device surface during removal.

The technologies described in the present disclosure can provide the following technical advantages. First, the described differential adhesive systems are capable of adhering to a first side or surface of a cover with a low surface energy and supporting the cover upon another surface, such as that of a device. Second, the described adhesion systems are capable of supporting a cover on the surface for prolonged periods of time, such as overnight or over a weekend. Furthermore, the adhesion system allows the cover and adhesives to be cleanly removed after such prolonged periods (i.e., a 24 hour dwell) without leaving residue on the surface.

The technologies described in the present disclosure therefore provides a secure attachment without leaving behind residue on the machine that will collect dust and dirt, which is not desired, especially in a setting. Additionally, these technologies eliminate or reduce the need for and exposure to chemicals and solvents for removing adhesive residue, which require special storage requirements and additional labor. Additional details of implementations are now described in relation to the Figures.

### Example Embodiments

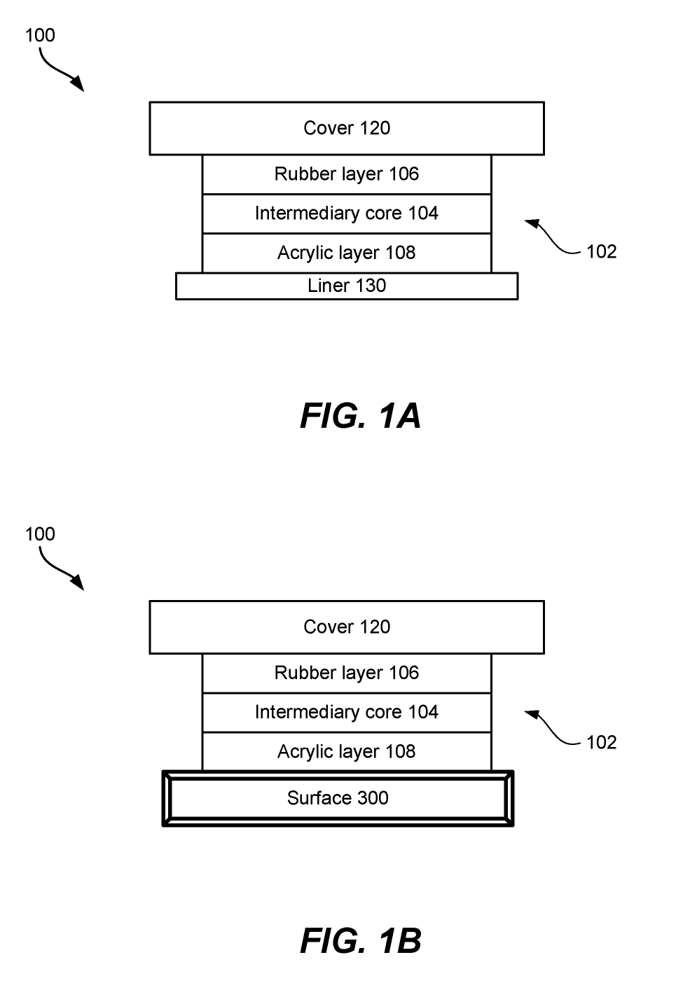

The present disclosure describes a differential adhesive system comprising a removable acrylic adhesive layer and a permanent rubber adhesive layer. FIGS. 1A and 1B illustrate an enlarged fragmentary vertical section of a differential adhesive system 100, in accordance with one or more embodiments. As illustrated in FIGS. 1A and 1B, a differential adhesive system 100 includes differential adhesive 102 comprising intermediary core 104, rubber layer 106 and acrylic layer 108. In various embodiments, differential adhesive system further includes cover 120.

In various embodiments, intermediary core 104 comprises a polyester film. In other embodiments, intermediary core 104 may comprise a polyethylene film. As used herein, intermediary core may be referred to herein as a “carrier” or “carrier film” or “intermediary film” or “intermediary body” or “intermediary core layer.” Rubber layer 106 and acrylic layer 108 are located on opposite sides of the intermediary core layer 104. This construction may be manufactured by coating one side of the intermediary core layer 104 with adhesive, laminating in a liner, then coating the opposite side of the intermediary core layer 104. Alternatively, one of the adhesives could be coated onto a release liner and that adhesive subsequently laminated to the opposite side of the previously adhesive coated intermediary core layer 104.

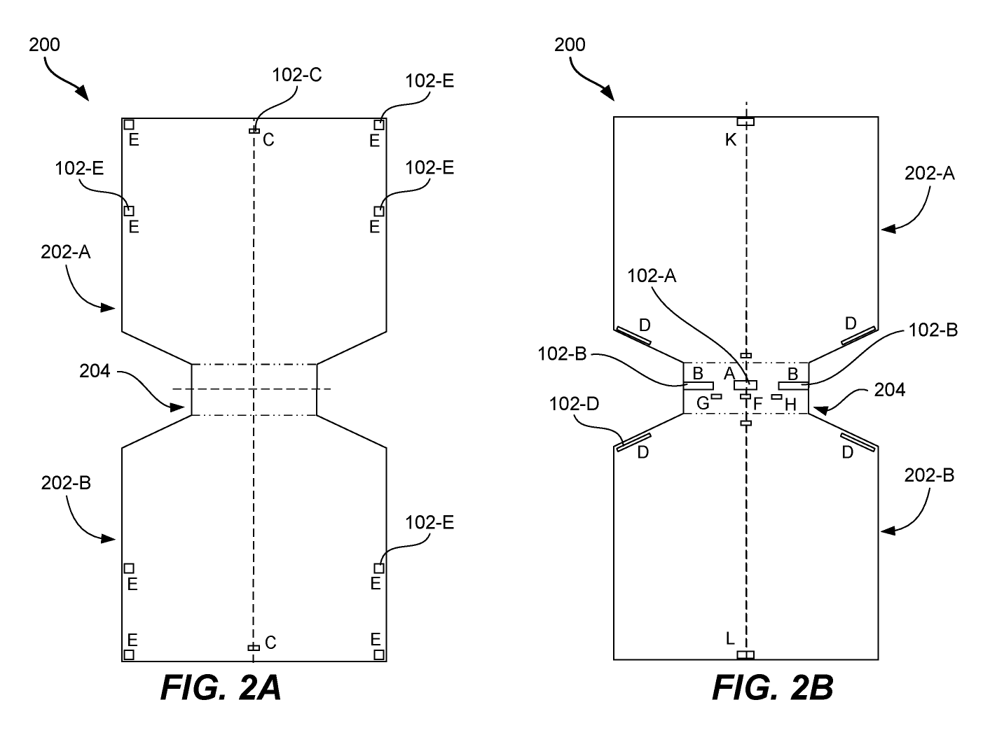

In various embodiments, rubber layer 106 is a permanent rubber adhesive applied to a first side of the intermediary core layer 104. In some embodiments, rubber layer 106 is a pressure sensitive rubber adhesive. Rubber layer 106 may be aggressive enough to adhere to a surface with low surface energy, such as protective cover 120. Rubber layer 106 may be applied to protective cover 120 via lamination to the cover. In various embodiments, cover 120 may be fabricated from a low surface energy plastic film, such as low density polyurethane (LDPE) plastic. Protective cover 200 is further described with reference to FIGS. 2A and 2B.

In some embodiments, the pressure sensitive rubber adhesive of rubber layer 106 may comprise a styrene-butadiene-styrene (SBS) block copolymer. The rubber adhesive may comprise a higher carbon-carbon double bond content primarily due to higher aromatic ring content (from styrene), as well as C5 aliphatic tackifier resin (i.e. cis/trans-1,3-pentadiene and cyclopentene).

In some embodiments, rubber layer 106 may include a peel adhesion of approximately 107 ounces per inch (oz/in), or 6.7 pound-force per inch (lbf/in). In some embodiments, rubber layer 106 may include a peel adhesion of approximately 100 to 120 ounces per inch (oz/in). In yet other embodiments, the rubber layer 106 may include a peel adhesion of at least 90 oz/in or higher. A rubber layer with a peel adhesion of lower than 90 oz/in may result in adhesive confusion, making it difficult to remove the liner, or causing the rubber layer 106 to completely delaminate from cover 200 during cover removal. Such peel force may be measured based on a 180 degree peel test to a standard steel panel using test standard ASTM D-3330—STD 10 from the American Section of the International Association for Testing Materials, or the test standard PSTC-101 from the Pressure Sensitive Tape Council.

In various embodiments, acrylic layer 108 is a removable acrylic adhesive applied to an opposite second side of the intermediary core layer 104. FIG. 1A shows a differential adhesive system prior to application on a desired surface 300. As such, adhesive system 100 in FIG. 1A includes liner layer 130 covering acrylic layer 108. In various embodiments, liner layer 130 is configured to protect the adhesive characteristics of acrylic layer 108. In various embodiments, liner layer 130 may comprise a polyester film, such as polyethylene terephthalate (PET), polypropylene, polyethylene, etc. In other embodiments, liner layer 130 may comprise a paper release liner, such as glassine paper, super calendared kraft paper, clay coated kraft paper, etc. In some embodiments, liner layer 130 may include a low surface energy release coating such as a crosslinked silicone layer. In some embodiments, liner layer 130 may include a cured coating to further protect acrylic layer 108 from ultraviolet (UV) radiation. In some embodiments, liner layer 130 may be oversized with one or more dimensions being greater than the dimensions of differential adhesive 102. Such oversized configuration allows for easier removal of liner layer 130. In some embodiments, liner layer 130 may be marked or otherwise colored differently from that of cover 120 for increased visibility of the location of adhesive 102.

FIG. 1B shows a differential adhesive system applied to a desired surface 300. Acrylic layer 108 may be configured to removably adhere to surface 300. Surface 300 may be the surface of a device. In various embodiments, the device may be medical equipment (large stationary machinery), such as a CT scanner or MRI machine, etc. However, in some embodiments, the device may be smaller machinery, such as computer screens, control panels, mobile machinery, overhead lights, lamps, hand-held equipment such as surgical tools, etc. In some embodiments, surface 300 comprise a painted metal or plastic surface. In some embodiments, surface 300 may be a textured surface.

In some embodiments, acrylic layer 108 is a pressure sensitive acrylic adhesive. In some embodiments, acrylic layer 108 includes a “medium level of adhesion” as defined by a peel force to a stainless steel panel using test standard ASTM D-3330—STD 10 from the American Section of the International Association for Testing Materials, or the test standard PSTC-101 from the Pressure Sensitive Tape Council. For example, particular example embodiments, of acrylic layer 108 may include a peel adhesion of approximately 40 to 60 ounces per inch (oz/in) according to ASTM D-3330—STD 10—180 degree—12″ per minute to stainless steel, or according to PSTC-101—180 degree peel test to standard steel panel. In some embodiments, acrylic layer 108 may include a peel adhesion of approximately 51 oz/in, or 3.2 lbf/in. Acrylic adhesives with a peel adhesion of less than 40 oz/in may not possess sufficient bond strength to secure a cover a certain weight and size on the surface of a device. Acrylic adhesives with a peel adhesion of greater than 60 oz/in may be too aggressive and leave residual adhesive when removed from the surface of a device.

In some embodiments, acrylic layer 108 may comprise a thermoset cross-linked acrylic adhesive. The cross-linked configuration may allow acrylic layer 108 to maintain its structural integrity and prevent cracks and splits in layer 108 during removal from surface 300, preventing residue from being left behind on surface 300. However, in other embodiments, the acrylic adhesive may not be crosslinked. In some embodiments, an acrylic adhesive that is not crosslinked may include a higher glass transition temperature than an acrylic adhesive that is crosslinked. Acrylic layer 108 may exhibit carbonyl characteristics and/or alkyl characteristics.

In various embodiments, differential adhesive system 100 maintains its adhesive properties for a desired period of time. For example, adhesive system 100 may maintain its adhesive properties for at least 40 days when stored at 50° F.-80° F. (10° C.-27° C.) and at a relative humidity of 40-60 percent. The described adhesives also provide a more cost-effective solution than various silicone adhesives.

### Device Cover

As previously described, adhesive system 100 includes a cover 120 with differential adhesive 102. In some embodiments, cover 120 may include one or more patches or strips of differential adhesive 102. FIGS. 2A and 2B illustrate an example device cover 200 that may be implemented with various embodiments of the present disclosure. In various embodiments, device cover 200 may be cover 120. FIG. 2A depicts a bottom view of cover 200, while FIG. 2B depicts a top view of cover 200. Various examples of device covers are further described in U.S. Provisional Patent Application Ser. No. ______, entitled REMOVABLY ATTACHABLE PROTECTIVE GANTRY COVER (Attorney Docket No. PDC1P005P) by Jennifer Thompson et al., filed on March 2018, which application is incorporated by reference herein in its entirety and for all purposes.

Protective cover 200 may comprise various flexible or rigid plastic or plastic-like materials. In various described embodiments, protective cover 200 may be a waterproof or water resistant material. For example, protective cover 200 may comprise various plastic materials, including polyvinyl film, polyurethane, polyethylene, etc. In an example embodiment, protective cover 200 is fabricated from low density polyethylene (LDPE) plastic.

In another example use, the plastic cover with integrated pressure sensitive adhesive can be used for covering parts during product manufacture or maintenance where prolonged but temporary use of a cover is needed, and where the adhesive needs to remove cleanly from the surface. For instance, currently, vehicle frames are covered with woven barriers during manufacture, and plastic coverings are taped onto the vehicle frames during body work. Woven barriers are generally more expensive than plastic barriers. Furthermore, the costs for labor and time for the taping of such plastic coverings would be greatly reduced.

In an example embodiment, cover 200 may include a tensile property durable enough to withstand the weight of secretions or fluids up to 2.5 pounds. However, cover 200 may be configured to withstand greater or lesser weight depending on various user requirements. Such tensile properties may be measured according to ASTM D882 standards.

In an example embodiment, cover 200 is translucent such that device patient positioning colored lasers, or other optical mechanisms, can transmit clearly through the cover. In some embodiments, cover 200 is additionally configured to be radiolucent or transparent in images captured by the device, and do not affect image quality. As such, cover 200 would allow for an artifact-free image without requiring increased radiation during operation, thereby preventing increased patient radiation dosage.

Protective cover 200 may be a cover configured to create a protective barrier against fluids and other debris from damaging or soiling a device, such as Computed Tomography (CT) machines, Magnetic Resonance Imaging (MRI) machines, and other imaging devices. Protective cover 200 may additionally or alternatively function to create a sterile barrier between a patient and the device. As such, protective cover 200 may protect valuable equipment from potential damage and decrease clean-up time when fluid spills or issues occur. For example, cover 200 may provide protection against viral penetration as measured using ASTM F1671/F167M—13 Standard Test Method for Resistance of Materials Used in Protective Clothing to Penetration by Blood-Borne Pathogens Using Phi-X174 Bacteriophage Penetration as a Test System.

For example, protective cover 200 may be configured to line the interior of an imaging device. As another example, protective cover 200 may be configured to protect the bottom of a CT gantry (center opening) and scanner from fluids that may enter into the imaging window or spill down the front table side of the scanner or back non-table side of the scanner. In various embodiments, protective cover 200 may be a cover configured to line various other devices, such as gurneys, operating tables, examination chairs and benches, etc. In some embodiments, protective cover 200 may be configured to cover the exterior surface of various devices and/or equipment to protect from dust, fluids, and debris.

In various embodiments cover 200 may include a symmetrical shape, as depicted, to provide equal coverage of both table and non-table sides of a device, such as a CT scanner. Cover 200 may be configured to fit at least one CT scanner approved by the U.S. Food and Drug Administration (FDA). Cover 200 may include an extended drape size to provide full protection to both the table and non-table sides of the bottom of the CT gantry opening and scanner. Coverage may extend to the floor, reducing fluids getting under the scanner or on foot pedals. Cover 200 may be optimized to correct poor adherence to an inner bore of the CT scanner during installation. Cover 200 may also be configured to be folded in a manner for faster and easier unfolding than original covers. Folding technique changed to correct poor adherence to inner bore during installation.

In an example embodiment, cover 200 may include two symmetrical drape portions, 202-A and 202-B, connected by a narrow bore section 204. The total length of cover 200 may be approximately 170 inches with each drape portion extending approximately 77 inches and the bore section extending approximately 16 inches of the total length. The total width of the widest portion of each drape portion may be approximately 82 inches, while the corresponding dimension of the narrower bore section extends approximately 39 inches.

The cover 200 may be configured with one or more adhesives, such as adhesives 102 to removably secure cover 200 to a device, such as a CT scanner. As illustrated in FIGS. 2A and 2B, cover 200 includes one or more tabs or strips of adhesives 102-A, 102-B, 102-C, 102-D, and 102-E. Each of these described adhesives may be adhesive 102 with particular dimensions. On the top surface of cover 200, adhesive 102-A is located at location A on the bore section, adhesives 102-B is located at locations B on the bore section, and adhesive 102-D is located at locations D on each of the drape portions. On the bottom surface of cover 200, adhesives 102-C are located at locations C on the drape portions, while adhesives 102-E are located at locations E on the drape portions. Such configuration of adhesives on cover 200 may securely attach cover 200 to the surface of a device for at least 24 hours.

In some embodiments, such adhesives allow cover 200 to be adhered to the CT gantry bore during an imaging procedure. In some embodiments, cover 200 may include additional optional adhesive tabs or strips to allow for customizable protection based on the configuration of the device. For example, additional adhesive tabs may allow the cover to be pulled back over itself to improve fit or increase security of attachment to the device.

In some embodiments, the size of adhesive tabs or strips may be increased or the number of adhesive tabs or strips may be decrease to allow for faster application. In some embodiments, adhesives may be covered with a colored liner layer, such as liner 130 for easier identification, location, and differentiation from other labeling on cover 200. Such labeling may include application instructions, such as those located at locations F, G, H, I, J, K, and L, as shown in FIGS. 2A and 2B. As previously described, such liner 130 may be oversized to create a section not secured to the adhesive to provide for convenient removal of the liner.

### Method of Operation

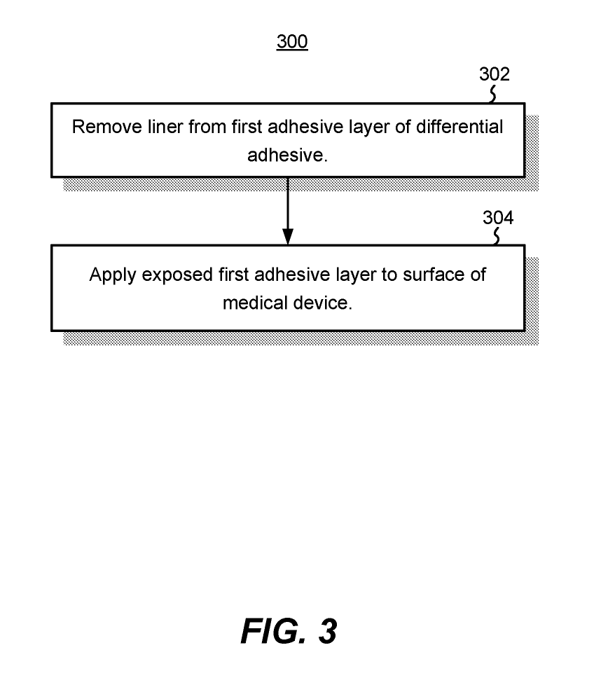

FIG. 3 illustrates an example method 300 for attaching a device cover to a surface of a device, in accordance with one or more embodiments. In various embodiments, the device cover is cover 200 with one or more adhesive strips or tabs, as described herein. Method 300 may comprise removing (302) a liner from a first adhesive layer of a differential adhesive, such as differential adhesive 102. For example, the liner may be liner 130 and the first adhesive layer may be acrylic layer 108. Method 300 may then comprise applying (304) the first adhesive layer to the surface of a device, such as a CT scanner, for instance.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first adhesive layer could be termed a second adhesive layer, and, similarly, a second adhesive layer could be termed a first adhesive layer, without changing the meaning of the description, so long as all occurrences of the “first adhesive layer” are renamed consistently and all occurrences of the “second adhesive layer” are renamed consistently. The first adhesive layer and the second adhesive layer are both adhesive layers, but they are not the same adhesive layer.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A differential adhesive for securing a plastic article to a surface of a first object, the differential adhesive comprising:
an intermediary body having a first side and an opposite second side;
a first adhesive layer attached to the first side, the first adhesive layer comprising an acrylic adhesive; and
a second adhesive layer attached to the second side, the second adhesive layer comprising a rubber adhesive secured to the plastic article.

2. The differential adhesive of claim 1, wherein the intermediary body comprises a polyester film.

3. The differential adhesive of claim 1, wherein the intermediary body comprises a polyethylene film.

4. The differential adhesive of claim 1, wherein the acrylic adhesive of the first adhesive layer includes a peel adhesion of approximately 40 ounces per inch to approximately 60 ounces per inch.

5. The differential adhesive of claim 1, wherein the acrylic adhesive of the first adhesive layer comprises a cross-linked pressure sensitive acrylic adhesive.

6. The differential adhesive of claim 1, wherein the first adhesive layer is configured for releasable attachment to the surface of the first object.

7. The differential adhesive of claim 1, wherein the second adhesive layer is configured for permanent attachment to the plastic article.

8. The differential adhesive of claim 1, wherein the plastic article is a device covering comprising polyethylene.

9. The differential adhesive of claim 1, wherein the plastic article is radiolucent.

10. The differential adhesive of claim 1, wherein the first object is a CT scanner or an MRI machine.

11. A device cover comprising:
a covering of plastic material;
a differential adhesive for securing the covering to a surface of a first object, the differential adhesive comprising:
an intermediary body having a first side and an opposite second side;
a first adhesive layer attached to the first side, the first adhesive layer comprising an acrylic adhesive;
a second adhesive layer attached to the second side, the second adhesive layer comprising a rubber adhesive secured to the covering.

12. The device cover of claim 11, wherein the intermediary body comprises a polyester film.

13. The device cover of claim 11, wherein the intermediary body comprises a polyethylene film.

14. The device cover of claim 11, wherein the acrylic adhesive of the first adhesive layer includes a peel adhesion of approximately 40 ounces per inch to approximately 60 ounces per inch.

15. The device cover of claim 11, wherein the acrylic adhesive of the first adhesive layer comprises a cross-linked pressure sensitive acrylic adhesive.

16. The device cover of claim 11, wherein the first adhesive layer is configured for releasable attachment to the surface of the first object.

17. The device cover of claim 11, wherein the second adhesive layer is configured for permanent attachment to the covering.

18. The device cover of claim 11, wherein the first object is a CT scanner or an MRI machine.

19. The device cover of claim 11, wherein the covering comprises a polyethylene film.

20. A method of attaching a device cover to a surface of a device, the method comprising:
removing a liner from a differential adhesive secured to the device cover, wherein the differential adhesive comprises:
an intermediary body having a first side and an opposite second side;
a first adhesive layer attached to the first side, the first adhesive layer comprising an acrylic adhesive covered by the liner;
a second adhesive layer attached to the second side, the second adhesive layer comprising a rubber adhesive secured to the covering; and
applying the exposed first adhesive layer to the surface of the device.
